(19) United States
(12) Patent Application Publication
(10) Pub. No.: US 2006/0212023 A1
(43) Pub. Date: Sep. 21, 2006

(54) Locking drainage catheter

(76) Inventor: Jeffrey M. Cross, Boston, MA (US)

Correspondence Address: FAY KAPLUN & MARCIN, LLP, 15O BROADWAY, SUITE 702, NEW YORK, NY, 10038, US

(21) Appl. No.: 11/082,431

(22) Filed: Mar. 17, 2005

(51) Int. Cl. A61M 25/00

(52) U.S. Cl. 604/523

(57) ABSTRACT

A catheter comprises an elongated body extending from a curved, distal portion to a proximal portion and including a fluid transport lumen extending therethrough and a suture lock collar slidably coupled to the proximal portion for movement relative thereto. The suture lock collar includes a connection for coupling a fluid receiving structure to a proximal end of the catheter in combination with an actuation member connected to a distal end of the distal portion and extending through the catheter to couple to the suture lock collar so that movement of the suture lock collar proximally along the elongated body draws the suture proximally and curls the distal portion into a desired shape.

### BACKGROUND OF THE INVENTION

Catheters are routinely used to form a semi-permanent path into the body for the transfer of fluids to and from the body without having to repeatedly insert a needle into the patient. Such catheters are used, for example, to dispense therapeutic compounds into the body or to remove fluids, for example, to drain fluids caused by infection, trauma, abscess or normal metabolic function such as urinating.

Pigtail catheters which are maintained straight during insertion and which, upon deployment, curl into an annular shape, are especially useful for the drainage of abscesses or the urinary bladder. The pigtail construction promotes retention of the catheter in the target location (e.g., within the bladder) as the size of the curled distal tip extends across the opening of the vessel through which the catheter was inserted into the location. The distal tips of conventional pigtail catheters are looped by pulling a string or suture extending from the distal tip along the catheter to a manual control at the proximal end. Thus, an additional step is required to deploy the distal end of the catheter after it has been positioned. The catheter is then connected to another device (e.g., a collection bag, additional tubing) using a pair of locking hubs.

### SUMMARY OF THE INVENTION

In one aspect, the present invention is directed to a catheter comprising a flexible elongated body extending from a curved distal portion to a proximal portion and including a fluid transport lumen extending therethrough and a suture lock collar slidably coupled to the proximal portion for movement relative thereto, the suture lock collar including a connection for coupling a fluid receiving structure to a proximal end of the catheter in combination with a suture connected to a distal end of the flexible distal portion, the suture extending internally through the catheter to couple to the suture lock collar so that movement of the suture lock collar proximally along the elongated body draws the suture proximally and curls the flexible distal portion into a desired shape.

A further aspect of the present invention is directed to a method for deploying a catheter, comprising inserting a distal portion of the catheter into the body while maintaining the shape of a curved, distal end of the catheter substantially straight and, after the distal end of the catheter has reached a desired position within a hollow organ, moving a first lock collar of the catheter proximally to connect the catheter to a second lock collar of a fluid receiving device, the catheter including an actuation member extending therethrough between the distal end and the first lock collar so that, when the first lock collar is moved proximally to connect to the second lock collar, the distal end is pulled into a desired, curved shape to anchor the flexible, distal end in the hollow organ.

### BRIEF DESCRIPTION OF THE DRAWINGS

FIG. 1 is a top plan view showing a according to an embodiment of the invention with the distal end formed into a pigtail;

FIG. 2 is a side elevation view of the catheter shown in FIG. 1;

FIG. 3 is a cross sectional view with an enlarged view of the catheter shown in FIG. 2, along line III-III;

FIG. 4 is a side view showing a catheter according to a further embodiment of the invention;

FIG. 5 is a side view of the catheter of FIG. 4 showing a locking arrangement of the catheter and a second device coupled thereto; and

FIG. 6 is a cross-sectional view of a suture lock collar of the catheter of FIG. 4.

### DETAILED DESCRIPTION

The present invention may be further understood with reference to the following description and the appended drawings, wherein like elements are referred to with the same reference numerals. The invention is related to catheters for draining fluids from, and infusing fluids, into the body.

Catheters are often formed with an outer shell formed of a biocompatible material impermeable to the fluid flowing therein. Generally, a flexible material is used, to reduce patient discomfort during insertion and during subsequent movement of the patient.

According to the present invention, a pigtail catheter is provided deployable by applying tension to a suture that is largely internal to the catheter, during and after deployment reducing the risk of infection and of degradation of the suture material over time and reducing leakage. The embodiments of the present invention also provide for a pigtail catheter in which the distal end is automatically curled into a desired pigtail shape as the locking hubs of the catheter and another device are joined together without the performance of additional acts by the user.

An exemplary embodiment of the catheter according to the invention is shown in FIGS. 1-3. The catheter 100 is, for example, a drainage catheter, for example, a urinary catheter that is advanced into the bladder or into the renal pelvis to drain urine therefrom. In other embodiments, the catheter 100 may be used to drain abscesses or cysts. An appropriate size of the catheter 100 may be selected, depending on which application the catheter is used for.

The exemplary catheter 100 comprises a flexible, elongated body 106 that terminates at a distal portion 102. The distal portion 102 is biased toward a desired curled shape, for example, by heat setting. The elongate body 106 is generally kept substantially straight during insertion into the site to be drained, for example, using a stiffening member inserted therein. A suture 108 is internally attached near a distal tip 118 of the flexible portion 102 and extends through a lumen 101 extending through the catheter 100 substantially the length of the elongated body 106 to an opening 103 formed in a proximal end of the catheter. A plurality of holes 104 are formed in the wall of the flexible portion 102 to fluidly couple the lumen 101 to the external environment, facilitating the drainage of fluid from the area surrounding the flexible portion 102 through the lumen 101 to a separate fluid conduit or reservoir (not shown) attached to the proximal end of the catheter 100. These holes 104 may continue along any portion of the length of the elongated body 106 to tailor the catheter 100 to any specific application.

A suture lock collar 112 is slidably received around the proximal portion of the catheter for movement between a distal, de-coupled position and a proximal, coupled position in which the suture lock collar 112 may be engaged with a corresponding second lock collar 114 of a separate device to fluidly couple the lumen 101 to a corresponding lumen in the separate device. The suture 108 extends through the lumen 101 to an exit orifice 111 through which the suture 108 penetrates the wall of the catheter 100 to couple to the suture lock collar 112. The exit orifice is preferably located on a portion of the catheter 100 which, when the suture lock collar 112 is in the distal, de-coupled position, is covered by the suture lock collar 112. This portion of the catheter 100 is designed to remain outside the body when the flexible portion 102 is in a desired position within the body. Thus, moving the suture lock collar 112 proximally from the de-coupled position to the coupled position to couple the catheter 100 to a separate device, draws the suture proximally pulling the flexible section 102 into the desired curved, pigtail shape.

As described above, the suture lock collar 112 is slidable longitudinally along the elongated body 106 between the coupled and de-coupled positions and includes a connection end 122 adapted to form a fluid tight connection with a second lock collar 114 of a separate device as described above. The second lock collar 114 may include, for example, a connection end 124 adapted to form a connection with the suture lock collar 112 to maintain a fluid connection between the lumen 101 and a luer 116 or other connection leading to additional tubing, a drainage bag or other device as would be understood by those of skill in the art. Those skilled in the art will understand that the locking hubs 112, 114 may have, for example, a mechanical coupling or friction fit so that they may be easily snapped together to form the connection.

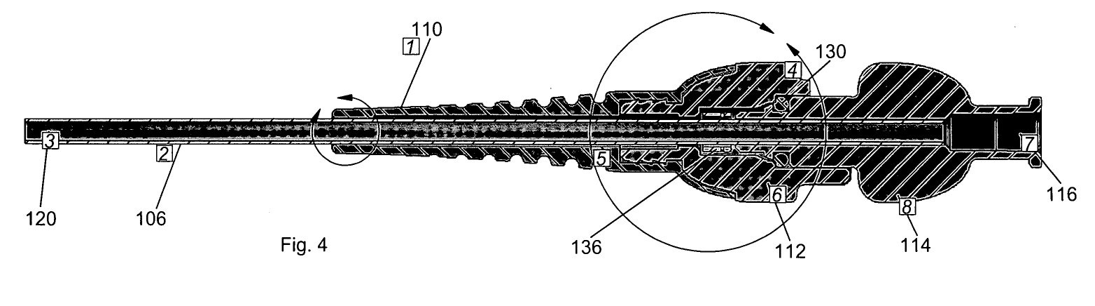

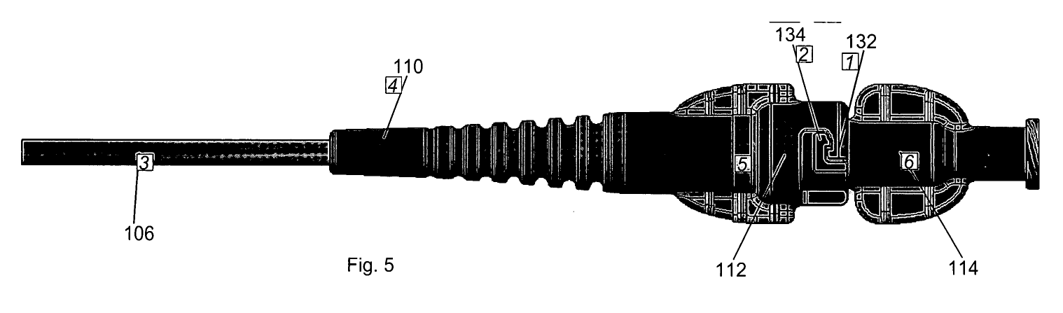

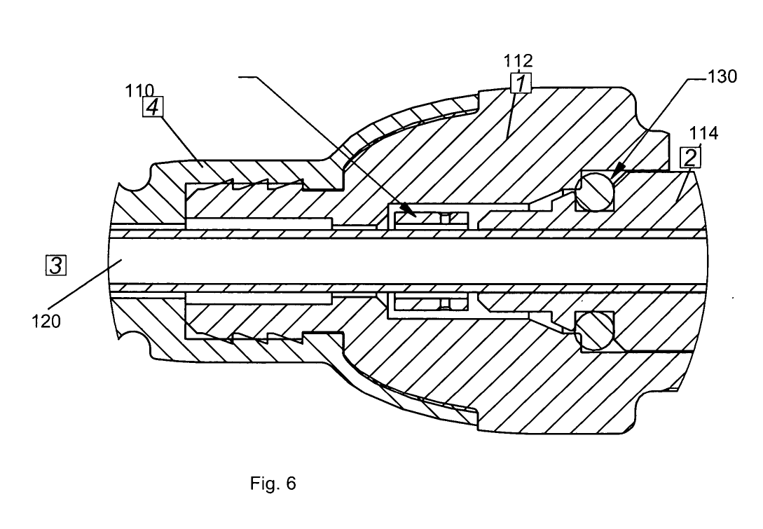

As shown in FIGS. 4-6, the second lock collar 114 may include an O-ring 130 received within a channel near a distal end thereof. The O-ring 130 provides a seal between the suture lock collar 112 and the second lock collar 114 so that any fluids which may leak out of the exit orifice 111 are prevented from escaping to the exterior of the suture lock collar 112 and/or the second lock collar 114. In addition, as the suture lock collar 112 is pushed proximally into the second lock collar 114, the O-ring 130 is compressed slightly. When the pressure against the O-ring 130 is released it expands to maintain a locking arm 132 of the suture lock collar 112 in a locking position within a mating arm 134 of the second lock collar 114. In addition, FIGS. 4 and 6 show the suture collar 136 to which the suture 108 is coupled. Those skilled in the art will understand that, as the suture collar 136 is received within the suture lock collar 112, movement of the suture lock collar 112 proximally draws the suture collar 136 and the suture 108 attached thereto proximally. The bias of the distal portion 102 to the curved position prevents the suture collar 136 from moving proximally out of the suture lock collar 112.

As shown in FIG. 1, a short segment of the suture 108 extends out of the elongated body 106 through the exit orifice 1 11 to the suture lock collar 1 12. As described above, when the suture lock collar 112 is in the distal, de-coupled position, the suture lock collar 112 completely covers this exposed portion of the suture 108. Even when moved proximally to the coupled position and the suture lock collar 112 is moved away from the exit orifice 111, only the portion of the suture 108 extending from the exit orifice 111 to the distal end of the suture lock collar 112 is uncovered. Thus, the length of the suture 108 exposed to the environment outside the catheter 100 is minimized and is less than a distance the suture lock collar 112 is able to translate longitudinally along the elongated body 106, as will be described below.

A flexible, strain relief section 110 or sheath may also be provided around the proximal portion of the catheter to form a seal around the exposed portion of the suture 108 to prevent fluid leakage from the orifice 111 from contaminating the surfaces of the catheter 100 which are handled by health care professionals, etc. The flexible, strain relief section 110 may, for example, be formed as a shell-like structure a distal end of which creates a fluid tight seal around a portion of the elongated body 106 immediately distal to a distal end of the suture lock 112 when the suture lock 112 is in the de-coupled position. A proximal end of the strain relief section is preferably coupled to the distal end of the suture lock collar 112 so that, as the suture lock collar 112 is moved proximally to the coupled position, the strain relief section 110 is drawn proximally to cover the exposed portion of the suture 108 while maintaining a seal around the entire area exposed to the suture 108 and any fluids leaking from the exit orifice 111. Thus, drawing the suture lock collar 112 proximally to couple the catheter 100 to a separate device draws the flexible section 102 into the desired curved, pigtail shape and pulls the strain relief section 110 proximally to ensure that the suture 108 is completely internal to the catheter 100 and is not exposed to the outside environment.

As shown in the enlargement of FIG. 3, the suture 108 is attached to the suture lock collar 112 at an attachment point 128. In this manner, when the suture lock collar 112 is moved proximally, the suture 108 is pulled along with it. As described above, a locking collar of a conventional pigtail catheter is generally translated proximally after insertion, to connect the catheter to another medical device, for example, a drainage bag. Thus, this movement in use of the catheter 100 which also effectuate connection to a separate device also draws the suture 108 which is coupled to the sliding collar 126 proximally curling the flexible portion 102 into the pigtail shape without the separate step required with prior devices.

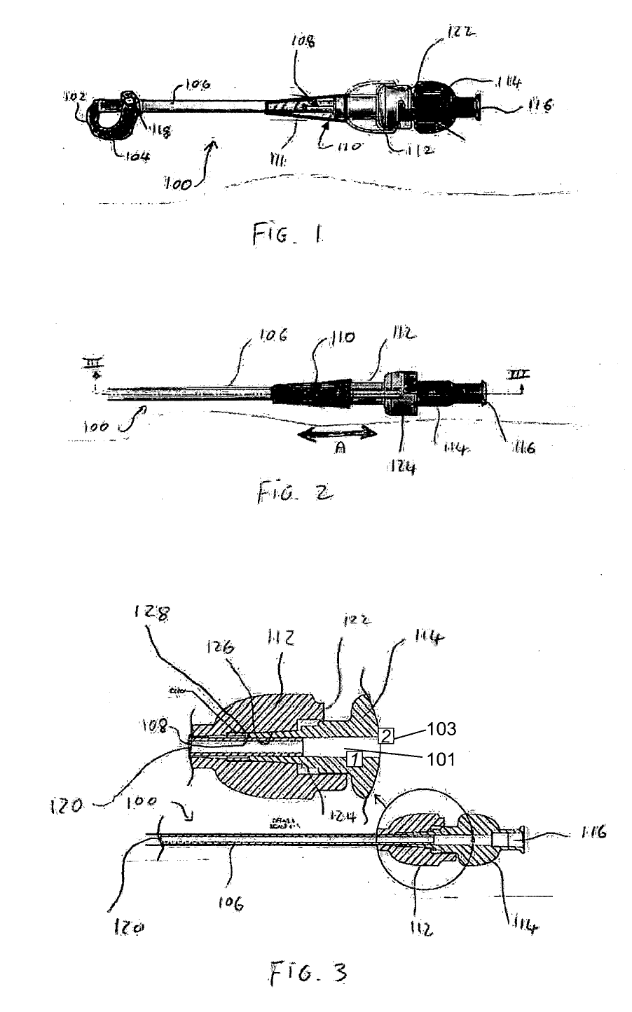

The use of the catheter 100 is described below with reference to FIGS. 1 and 2. The insertion of the catheter 100 proceeds in a generally conventional manner, with the suture lock collar 112 in the distal, de-coupled position spaced from the proximal end of the catheter 100 toward the flexible portion 102. At this point the second locking collar 114 has not yet been connected to the suture lock collar 112. After the catheter 100 has been properly positioned, e.g., with the flexible portion 102 extending past an opening from a small diameter vessel through which the catheter 100 was introduced into a larger organ, the user slides the suture lock collar 112 proximally in the direction of arrow A bringing the suture lock collar 112 nearer to the proximal end of the catheter 100 for connection to the second lock collar 114 to establish a fluid connection between the catheter 100 and another medical device, such as a drainage bag.

As described above, this proximal motion of the suture lock collar 112 applies tension to the suture 108, which in turn pulls the distal tip 118 inward, and curls the flexible portion 102 to form the pigtail shape within the larger hollow organ. The curled flexible portion 102 then provides a check to removal of the catheter 100 from the large organ as the flexible portion 102 is preferably curled into a shape of larger diameter than the vessel via which the catheter 100 was inserted into the organ. Thus, no separate action needs to be performed to secure the catheter in place, as the pigtail shape is formed automatically when the suture lock collar 112 is connected to the second lock collar 114. Then, when the suture lock collar 112 has been connected to the second lock collar 114, it is no longer able to move distally in the direction of the arrow A and the tension remains on the suture 108 which effectively locks the flexible portion 102 in the curled, pigtail shape preventing unintentional removal of the catheter 100 from the desired location.

The present invention has been described with reference to specific embodiments, and more specifically to a urinary drainage catheter. However, other embodiments may be devised that are applicable to other types of catheters, principally to drainage catheters. Accordingly, various modifications and changes may be made to the embodiments, without departing from the broadest spirit and scope of the present invention as set forth in the claims that follow. The specification and drawings are accordingly to be regarded in an illustrative rather than restrictive illustrative rather than restrictive sense.

What is claimed is:

1. A catheter comprising:
a flexible elongated body extending from a curved distal portion to a proximal portion and including a fluid transport lumen extending therethrough;
a suture lock collar slidably coupled to the proximal portion for movement relative thereto, the suture lock collar including a connection for coupling a fluid receiving structure to a proximal end of the catheter; and
a suture connected to a distal end of the flexible distal portion, the suture extending internally through the catheter to couple to the suture lock collar so that movement of the suture lock collar proximally along the elongated body draws the suture proximally and curls the flexible distal portion into a desired shape.

2. The catheter according to claim 1, further comprising a plurality of holes extending through a wall of the distal portion to the lumen.

3. The catheter according to claim 2, further comprising at least one hole extending through a wall of the proximal portion of the elongated body.

4. The catheter according to claim 1, wherein the elongated body is formed of a flexible material and the distal portion is formed integrally with the proximal portion of the elongated body.

5. The catheter according to claim 1, wherein the suture extends from the distal end of the distal portion through the lumen to the suture lock collar.

6. The catheter according to claim 3, further comprising an exit orifice extending out of the catheter from the lumen, the suture extending through the exit orifice to couple to the suture lock collar.

7. The catheter according to claim 4, further comprising a sheath sealing around a portion of the catheter including the exit orifice.

8. The catheter according to claim 7, wherein the sheath is flexible.

9. The catheter according to claim 7, wherein the sheath is attached to the suture lock collar.

10. The catheter according to claim 9, wherein a proximal end of the sheath is attached to a distal end of the suture lock collar.

11. The catheter according to claim 1, wherein, when the suture lock collar is in a proximal position for coupling to a separate device, a tension applied to the suture maintains the distal portion curled in the desired shape.

12. The catheter according to claim 1, wherein the distal portion is biased toward the desired shape.

13. A catheter comprising:
a flexible elongated body extending from a curved distal portion which, when in an operative position, resides within a hollow organ of a body to a proximal portion which remains outside the body;
a lock collar slidably mounted on the proximal portion for movement between a distal, de-coupled position and a proximal, coupling position for fluidly coupling a proximal end of lumen to a separate device via a connector of the lock collar; and
an actuation member extending from the lock collar to a distal end of the distal portion so that, when the lock collar is moved from the distal, de-coupled position to the proximal, coupling position, the actuation member pulls the distal end of the distal portion to curl the distal portion into a desired shape to anchor the distal portion within the hollow organ.

14. The catheter according to claim 13, wherein the actuation member extends between the distal end of the distal portion and the lock collar within the catheter.

15. The catheter according to claim 13, the catheter including a fluid lumen extending therethrough between the proximal portion and the distal portion, wherein the actuation member extends from the distal end of the distal portion through the lumen to an exit orifice which, when the lock collar is in the distal, de-coupled position, is covered by the lock collar.

16. The catheter according to claim 15, further comprising a flexible sheath sealing an area around the exit orifice.

17. The catheter according to claim 16, wherein a distal end of the sheath is sealed around a portion of the catheter distal to the exit orifice and a proximal end of the sheath is coupled to a distal end of the lock collar.

18. The catheter according to claim 13, wherein the distal portion of the catheter includes a plurality of holes communicating with the lumen.

19. A method for deploying a catheter, comprising:
inserting a distal portion of the catheter into the body while maintaining the shape of a curved, distal end of the catheter substantially straight;
after the distal end of the catheter has reached a desired position within a hollow organ, moving a first lock collar of the catheter proximally to connect the catheter to a second lock collar of a fluid receiving device, the catheter including an actuation member extending therethrough between the distal end and the first lock collar so that, when the first lock collar is moved proximally to connect to the second lock collar, the distal end is pulled into a desired, curved shape to anchor the flexible, distal end in the hollow organ.

20. The method according to claim 19, wherein moving the actuation member comprises a length of suture and wherein proximal movement of the first lock collar applies tension to the suture.

21. The method according to claim 20, wherein connecting the first and second lock collars to one another locks the distal end in the desired shape.
